(19) United States
(12) Patent Application Publication
(10) Pub. No.: US 2008/0226983 A1
(43) Pub. Date: Sep. 18, 2008

(54) NON-AQUEOUS ELECTROLYTE AND NON-AQUEOUS ELECTROLYTE BATTERY USING THE SAME

(75) Inventors: Toru Odani, Fukushima (JP); Tadahiko Kubota, Kanagawa (JP)

Correspondence Address: BELL, BOYD & LLOYD, LLP, P. O. BOX 1135, CHICAGO, IL, 60690, US

(73) Assignee: SONY CORPORATION, Tokyo (JP)

(21) Appl. No.: 12/048,999

(22) Filed: Mar. 14, 2008

(30) Foreign Application Priority Data
Mar. 16, 2007 (JP) P2007-069113
Jan. 31, 2008 (JP) P2008-020773

(51) Int. Cl. H01M 10/40; C07F 7/18; C07D 319/14; C07C 49/753; C07C 49/707

(52) U.S. Cl. 429/200; 429/188; 429/199; 556/436; 568/379; 568/377; 568/381; 549/362

(57) ABSTRACT

The invention provides a non-aqueous electrolyte including: at least one kind selected from the compounds represented by Chemical formula 1:

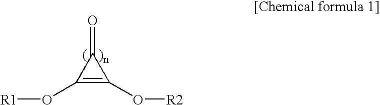

wherein R1 and R2 each represent a hydrogen group, a hydrocarbon group or an alkylsilyl group, or any of these groups part or all of the hydrogen atoms of which is/are substituted with a halogen, R1 and R2 may be bonded to each other, and n is an integer of 1 or larger.

### CROSS REFERENCE TO RELATED APPLICATIONS

The present application claims priority to Japanese Patent Application No. 2007-069113 filed on Mar. 16, 2007 and Japanese Patent Application No. 2008-020773 filed on Jan. 31, 2008, the entire contents of which are being incorporated herein by reference.

### BACKGROUND

This present application relates to a non-aqueous electrolyte and a non-aqueous electrolyte battery using the same, and more specifically to a non-aqueous solvent, a non-aqueous electrolyte including an electrolyte salt and a non-aqueous electrolyte battery using the same.

Lithium ion secondary batteries are secondary batteries making use of occlusion and discharge of lithium ions for the charge and discharge reaction, and greatly expected since the batteries can obtain a large energy density as compared with lead batteries and nickel-cadmium batteries.

Lithium ion secondary batteries widely utilize carbon materials as anode active materials thereof. For example, proposed are lithium ion secondary batteries that use carbon materials capable of occlusion and discharge of lithium ions of cokes, artificial graphite, natural graphite and the like. In such lithium ion secondary batteries, lithium is not present in a metal form, so that the formation of dendrites is suppressed to enables improvement of the battery life and safety. In particular, graphite-based carbon materials such as artificial graphite and natural graphite are expected as materials capable of improving an energy density per volume.

In a lithium ion secondary battery using a graphite-based carbon material alone for an anode, or in a lithium ion secondary battery using for an anode a mixture of a graphite-based carbon material and another anode material capable of occlusion and discharge of lithium, carbonate ester generally preferably used for a lithium primary battery is employed for a solvent of an electrolyte solution. However, when carbonate ester is used as a solvent of an electrolyte solution, the electrolyte solution decomposes on an electrode surface in a charge and discharge process, which poses problems of a decrease in charge and discharge efficiency and a decrease in cycle characteristics, and the like.

Thus, for suppression of a decrease in charge and discharge efficiency and a decrease in cycle characteristics, etc, for example, Japanese Patent Application Laid-Open (JP-A) Nos. 5-744886 and 8-45545 propose an additive that decomposes on an electrode surface prior to the electrolyte solution to form a film, thereby restraining the decomposition of the solvent. Also proposed is, for example, a method of adding to an electrolyte solution a cyclic carbonate having an unsaturated group such as vinylene carbonate or (4-vinyl)ethylene carbonate.

Incidentally, recently, the battery capacity is requested to be further improved. For example, for the further improvement of the battery capacity, the use of silicon Si, tin Sn, or the like in place of a carbon material has been studied. Because the theoretical capacities of silicon Si (4199 mAh/g) and tin Sn (994 mAh/g) are extremely larger than the theoretical capacity of graphite (372 mAh/g), great improvement of the battery capacity can be expected.

For example, as described in International Publication No. 01/031724, a secondary battery using a thin film of silicon Si or tin Sn as an anode active material can obtain a discharge capacity since the secondary battery suppresses the pulverization of the anode active material even when lithium Li is occluded and discharged.

In addition, as a method of improving cycle characteristics in case of using as an anode active material a metal, element or alloy such as tin Sn or silicon Si capable of bonding to lithium, and compounds thereof, JP-A-2004-47131 proposes, for example, a method that allows an electrolyte solution to contain a cyclic or chain carbonate ester having a halogen as a constituent element. The reason why this method improves the characteristics is estimated to be that in an initial charging period, a high ionic permeability and high stability film is formed on the surface of an anode, thereby suppressing the decomposition reaction of the electrolyte solution.

However, when silicon Si or tin Sn is used as an anode active material, lithium Li becomes high in activity when occluded. For this reason, when as a solvent of an electrolyte solution, a high dielectric constant solvent such as cyclic carbonate ester is used in combination with a low viscosity solvent such as chain carbonate ester, there are the possibilities that the chain carbonate ester is liable to primarily decompose and also the lithium becomes inactive. In this case, unless the pulverization of an anode active material is sufficiently suppressed in the charge and discharge process, the charge and discharge efficiency is lowered, thus failing to obtain sufficient cycle characteristics and storage characteristics.

In JP-A-2004-47131, inclusion of cyclic or chain carbonate ester having a halogen as a constituent element in an electrolyte solution enables the suppression of the decomposition reaction of the electrolyte solution. However, when silicon Si or tin Sn is used as an anode active material, sufficient cycle characteristics are not obtained. Additionally, even when a carbon material is used as an anode material, it is desired to further improve cycle characteristics.

### SUMMARY

It is desirable to provide a non-aqueous electrolyte capable of improving cycle characteristics and a non-aqueous electrolyte battery using the non-aqueous electrolyte.

A first embodiment is a non-aqueous electrolyte containing at least one kind selected from compounds represented by Chemical formula 1:

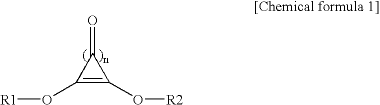

wherein R1 and R2 each represent a hydrogen group, a hydrocarbon group or an alkylsilyl group, or any of these groups part or all of the hydrogen atoms of which is/are substituted with a halogen, R1 and R2 may be bonded to each other, and n is an integer of 1 or larger.

A second embodiment is a non-aqueous electrolyte battery including a cathode, an anode, and a non-aqueous electrolyte, wherein the non-aqueous electrolyte contains at least one kind selected from compounds represented by Chemical formula 2:

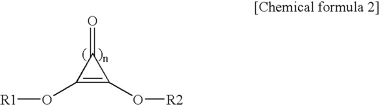

wherein R1 and R2 each represent a hydrogen group, a hydrocarbon group or an alkylsilyl group, or any of these groups part or all of the hydrogen atoms of which is/are substituted with a halogen, R1 and R2 may be bonded to each other, and n is an integer of 1 or larger.

In an embodiment, the compound represented by Chemical formula 1 contained in the non-aqueous electrolyte, when used for an electrochemical device such as a battery, can suppress the decomposition reaction of a solvent since the compound decomposes prior to the solvent to form a film on the top of an electrode.

According to an embodiment, a non-aqueous electrolyte is electrochemically stabilized to allow cycle characteristics to be improved.

Additional features and advantages are described herein, and will be apparent from the following Detailed Description and the figures.

### BRIEF DESCRIPTION OF THE FIGURES

FIG. 1 is a cross sectional view showing a configuration of a first example of a non-aqueous electrolyte battery using a non-aqueous electrolyte according to an embodiment;

FIG. 2 is a cross sectional view showing a configuration of a second example of a non-aqueous electrolyte battery using a non-aqueous electrolyte according to an embodiment;

FIG. 3 is a cross sectional view showing a part enlarged of a winded electrode member shown in FIG. 2;

FIG. 4 is a cross sectional view showing a configuration of a third example of a non-aqueous electrolyte battery using a non-aqueous electrolyte according to an embodiment; and

FIG. 5 is a cross sectional view taken along the line I-I of the winded electrode member shown in FIG. 3

### DETAILED DESCRIPTION

An embodiment of the present application will be described with reference to the drawings hereinafter. A non-aqueous electrolyte according to an embodiment is a non-aqueous electrolyte solution including, for example, a liquid solvent, an electrolyte salt dissolved in the solvent, and an oxocarbonic acid or an oxocarbonate derivative represented by Chemical formula 3:

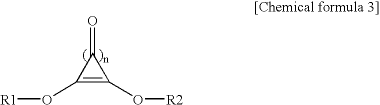

wherein R1 and R2 each represent a hydrogen group, a hydrocarbon group or an alkylsilyl group, or any of these groups part or all of the hydrogen atoms of which is/are substituted with a halogen, R1 and R2 may be bonded to each other, and n is an integer of 1 or larger.

The compound represented by Chemical formula 3 may decompose on an electrode to form a film. This film is estimated to be stable since electronic conjugation is extended on the entire ring of the oxocarbonic acid. This electrochemically stabilizes an electrolyte solution when the compound is used for batteries or the like, so that cycle characteristics can be improved.

In R1 and R2 in Chemical formula 3, examples of the hydrocarbon group include, specifically, saturated aliphatic hydrocarbon groups, unsaturated aliphatic hydrocarbon groups, aromatic hydrocarbon groups and alicyclic hydrocarbon groups, more specifically, methyl groups, ethyl groups, propyl groups, isopropyl groups, butyl groups, allyl groups, and phenyl groups. In addition, examples of the alkylsilyl group include specifically trialkylsilyl groups, and more specifically, trimethylalkylsilyl groups and triethylalkylsilyl groups.

As the compound represented by Chemical formula 3, a compound with n ranging from 1 to 6 in Chemical formula 3 is easily synthesized. More specifically, examples thereof include squaric acid or squaric acid derivatives such as 3,4-alkoxy-3-cyclobutene-1,2-dione, represented by Chemical formula 4; croconic acid or croconic acid derivatives such as 4,5-dialkoxy-4-cyclopentene-1,2,3-trione, represented by Chemical formula 5; and rhodizonic acid or rhodizonic acid derivatives such as 5,6-dialkoxy-5-cyclohexene-1,2,3,4-tetraone, represented by Chemical formula 6:

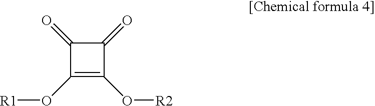

wherein R1 and R2 each represent a hydrogen group, a hydrocarbon group or an alkylsilyl group, or any of these groups part or all of the hydrogen atoms of which is/are substituted with a halogen, and R1 and R2 may be bonded to each other,

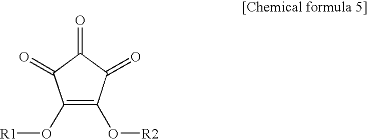

wherein R1 and R2 each represent a hydrogen group, a hydrocarbon group or an alkylsilyl group, or any of these groups part or all of the hydrogen atoms of which is/are substituted with a halogen, and R1 and R2 may be bonded to each other, and

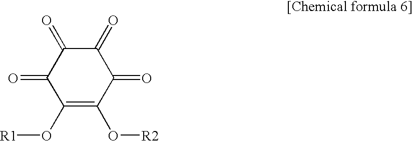

wherein R1 and R2 each represent a hydrogen group, a hydrocarbon group or an alkylsilyl groups, or any of these groups part or all of the hydrogen atoms of which is/are substituted with a halogen, and R1 and R2 may be bonded to each other.

The compound represented by Chemical formula 3 is preferably a compound of Chemical formula 3 in which R1 and R2 each represent an allyl group, a trialkylsilyl group such as a trimethylsilyl group, or a fluorine-substituted hydrocarbon group produced by substituting at least part of the hydrogen atoms of a hydrocarbon group such as “—CH2CF3” with fluorine. This is because the effect of further improving cycle characteristics can be obtained when a non-aqueous electrolyte containing a compound Chemical formula 3 in which R1 and R2 each represent an allyl group, a trialkylsilyl group, or a fluorine-substituted hydrocarbon group. Preferably, R1 and R2 are hydrocarbon groups from the viewpoint of obtaining excellent solubility, and more preferably R1 and R2 are different hydrocarbon groups from the viewpoint of obtaining more excellent solubility.

Available solvents include, for example, ethylene carbonate, propylene carbonate, butylene carbonate, dimethyl carbonate, diethyl carbonate, ethylmethyl carbonate, methylpropyl carbonate, γ-butyrolactone, γ-valerolactone, 1,2-dimethoxyethane, tetrahydrofuran, 2-methyltetrahydrofuran, tetrahydropyran, 1,3-dioxolane, 4-methyl-1,3-dioxolane, 1,3-dioxane, 1,4-dioxane, methyl acetate, ethyl acetate, methyl propionate, ethyl propionate, methyl butyrate, methyl isobutyrate, methyl trimethylacetate, ethyl trimethylacetate, acetonitrile, glutaronitrile, adiponitrile, methoxyacetonitrile, 3-methoxypropionitrile, N,N-dimethylformamide, N-methylpyrrolidinone, N-methyloxazolidinone, N,N′-dimethylimidazolidinone, nitromethane, nitroethane, sulfolane, trimethyl phosphate, and dimethylsulfoxide. This is because, in an electrochemical device including an electrolyte solution, such as a battery, excellent capacity characteristics, cycle characteristics and storage characteristics are obtained. These may be used alone or in combination of two or more thereof.

Of these, a preferably used solvent contains at least one kind selected from the group consisting of ethyl carbonate, propylene carbonate, dimethyl carbonate, diethyl carbonate and ethylmethyl carbonate because it exhibits a sufficient advantage. In this case, in particular, a solvent is preferably used that contains a mixture of ethylene carbonate or propylene carbonate as a high viscosity solvent (e.g., dielectric constant ∈≧30) and dimethyl carbonate, diethyl carbonate or ethylmethyl carbonate as a low viscosity solvent (e.g., viscosity≦1 mPa·s). This is because the dissociation properties of an electrolyte salt and the mobility of ions are improved to thereby obtain a higher effect.

Preferably, the solvent further contains a cyclic carbonate ester derivative such as 4-fluoro-1,3-dioxolane-2-one, or 4,5-difluoro-1,3-dioxolane-2-one, because cycle characteristics can be further improved.

Additionally, the solvent is preferable to further contain cyclic carbonate ester having an unsaturated bond such as vinylene carbonate (VC) or vinylethylene carbonate (VEC).

Moreover, the solvent is preferable to further contain a cyclic sultone derivative such as propane sultone or propene sultone (PRS).

Furthermore, the solvent preferably further contains an acid anhydride such as succinic anhydride or 2-sulfobenzoic anhydride.

Available examples of the lithium salts as the electrolyte salts include lithium hexafluorophosphate (LiPF6), lithium tetrafluoroborate (LiBF4), lithium perchlorate (LiClO4), lithium hexafluoroarsenate (LiAsF6), lithium trifluoromethanesulfonate (CF3SO3Li), lithium bis(trifluoromethanesulfonyl)imide [(CF3SO2)₂NLi](LiTFSI), lithium tris(trifluoromethanesulfonyl)methyl [(CF3SO2)₃CLi], lithium bisoxalate borate (LiBOB), and lithium 1,3-perfluoropropanedisulfonylimide. These may be used alone or in combination of two or more thereof.

A gel-like electrolyte prepared by causing a non-aqueous electrolyte solution to be held in a polymer compound may be used as the non-aqueous electrolyte. The gel-like electrolyte may have an ionic conductivity of 1 mS/cm or more at room temperature, and its composition and the structure of the polymer compound are not particularly limited. The solvent and electrolyte salt contained in the non-aqueous electrolyte and the compound represented by Chemical formula 3 are described above, and thus, detailed descriptions are omitted.

Available examples of the polymer compound include polyacrylonitrile, poly(vinylidene fluoride), copolymers of vinylidene fluoride and hexafluoropropylene, polytetrafluoroethylene, polyhexafluoropropylene, polyethylene oxide, polypropylene oxide, polyphosphazene, polysiloxane, poly(vinyl acetate), polyvinyl alcohol, poly(methyl methacrylate), polyacrylic acid, polymethacrylic acid, stylene-butadiene rubber, nitrile-butadiene rubber, polystyrene and polycarbonate. In particular, from the viewpoint of electrochemical stability, a polymer compound having a structure of polyacrylonitrile, poly(vinylidene fluoride), polyhexafluoropropylene, or polyethylene oxide is preferably used.

Note that the solvent used herein does not mean only a liquid solvent, and widely means a solvent capable of dissociating an electrolyte salt and having ionic conductivity. Thus, when an ion conducting solvent is used in a polymer compound, the polymer compound is also included in the solvent.

A non-aqueous electrolyte according to an embodiment can be suitably used for a non-aqueous electrolyte battery using as an anode a material containing at least one kind selected from silicon Si and tin Sn as a constituent element. This is because the battery using as an anode a material containing at least one kind selected from silicon Si and tin Sn as a constituent element has a high active anode to easily decompose its electrolyte, so that the cycle characteristics are prone to deteriorate as compared to the case where a carbon material is used for an anode, thereby obtaining a higher effect.

The particularly excellent effect of improving cycle characteristics can be obtained, in particular, in the case where a non-aqueous electrolyte containing a compound represented by Chemical formula 3 in which R1 and R2 each represent an allyl group, a trialkylsilyl group or a fluorine-substituted hydrocarbon is used for a non-aqueous electrolyte battery using as an anode a material containing at least one kind selected from silicon Si and tin Sn as a constituent element.

Furthermore, considering the battery characteristics, the compound represented by Chemical formula 3 is preferably contained at from 0.01 wt % to 10 wt % in the non-aqueous electrolyte. In addition, in the range of from 1 wt % to 5 wt %, substantially the same effect of improving cycle characteristics tends to be obtained.

For example, secondary batteries such as lithium batteries having a variety of shapes and sizes can be fabricated using the non-aqueous electrolyte according to one embodiment of the invention.

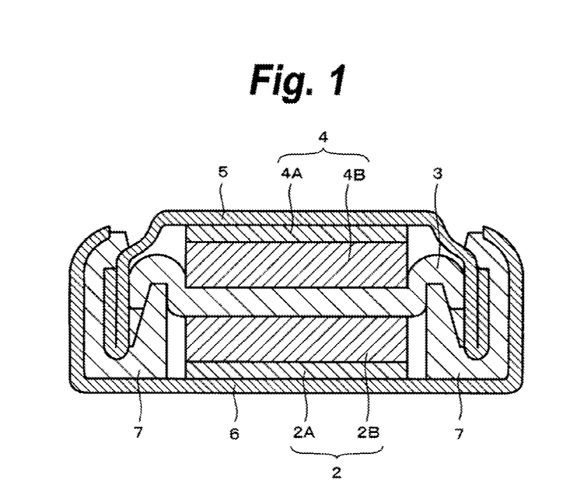

A first example of a non-aqueous electrolyte battery using the non-aqueous electrolyte according to one embodiment of the invention will be set forth. FIG. 1 shows one exemplary configuration of the non-aqueous electrolyte battery to one embodiment of the invention. This non-aqueous electrolyte battery has a so-called coin shape and, for example, a non-aqueous electrolyte secondary battery, e.g., a lithium ion secondary battery.

As shown in FIG. 1, this non-aqueous electrolyte battery includes a cathode 2, an outer covering can 6 which houses the cathode 2, an anode 4, an outer covering cup 5 which houses the anode 4, a separator 3 located between the cathode 2 and the anode 4, and a gasket 7 insulating the outer covering cup 5 from the outer covering can 6.

The cathode 2 has a structure in which a cathode active material layer 2B is placed on top of a cathode collector 2A. The cathode active material layer 2B contains, for example, as a cathode active material any one or two kinds of cathode materials capable of occlusion and discharge of lithium that is an electrode reactant, and may also include as necessary an electroconductive agent such as a carbon material and a binding agent such as poly(vinylidene fluoride).

Preferable examples of the cathode material capable of occlusion and discharge of lithium include lithium composite oxides such as lithium cobalt oxide, lithium nickel oxide or solid solutions containing these (Li(NixCoyMnz)O2: the values of x, y and z are 0<x<1, 0<y<1, 0<z<1 and x+y+z=1), and lithium manganese oxide (LiMn2O4) having a spinel structure or its solid solution (Li(Mn2-vNiv)O4: the value of v is v<2); and phosphate compounds having an olivine structure such as lithium iron phosphate (LiFePO4). This is because use of these materials can obtain an energy density. Besides the above cathode materials, the cathode materials include, for example, oxides such as titanium oxide, vanadium oxide or manganese dioxide, disulfides such as iron disulfide, titanium disulfide or molybdenum sulfide, sulfides such as sulfur, and electroconductive polymer such as polyaniline or polythiophene.

For example, a net-like or foil-like aluminum Al, etc. can be used as the cathode collector 2A. The binding agent used may be a well-known resin material, which is usually used for a non-aqueous electrolyte battery of this kind. More specifically, for example, poly(vinylidene fluoride) (PVdF) can be used. In addition, the electroconductive agent used may be one that is usually used for a non-aqueous electrolyte battery of this kind. More specifically, as the electroconductive agent used is, for example, carbon black or graphite.

The outer covering can 6 is a container made of an electroconductive metal, and is constituted by, for example, stainless SUS or aluminum Al.

The anode 4 has a structure in which an anode active material layer 4B containing an anode material capable of occlusion and discharge of lithium Li as an anode active material is formed on top of an anode current collector 4A.

Examples of the anode material capable of occlusion and discharge of lithium Li include carbon materials, metal oxides and polymer compounds. The carbon materials include, for example, graphiting carbon, non-graphiting carbon having the (002) plane with a surface separation of 0.37 nm or more, and graphite having the (002) plane with a surface separation of 0.340 nm or less. More specific examples include pyrolytic carbons, cokes, graphites, glass-like carbons, organic polymer compound baked bodies, carbon fibers and activated carbon. Of these, the cokes include pitch cokes, needle cokes, petroleum cokes, and the like. The organic polymer compound baked material refers to a carbonized material produced by baking a polymer compound such as a phenol resin or a furan resin at a suitable temperature. In addition, examples of the metal oxide include iron oxides, ruthenium oxides and molybdenum oxides, and examples of the polymer compound include polyacetylene and polypyrrole.

The anode active material layer 4B may contain, for example, as an anode active material at least one kind of anode materials selected from the group consisting of simple substances, alloys and compounds of metal elements capable of occlusion and discharge of lithium Li that is an electrode reactant, and simple substances, alloys and compounds of semi-metal elements capable of occlusion and discharge of lithium Li. This makes it possible to obtain a high energy density. Additionally, the anode material may be used together with the above-mentioned carbon material. The carbon material is preferable because it has a very small change in crystal structure during charge and discharge, and for example, when used with the above-mentioned anode material, can provide a high energy density and also excellent cycle characteristics, as well as functions as an electroconductive agent. Moreover, herein, the alloy includes, in addition to an alloy including two or more kinds of metal elements, an alloy including one or more kinds of metal elements and one or more kinds of semi-metal elements. Also, the alloy may include a nonmetallic element. Its composition may include solid solutions, eutectic crystals (eutectic crystal mixtures), inter-metal compounds or substances in which two or more kinds of them coexist.

Examples of the metal elements or semi-metal elements constituting the anode material include magnesium Mg, boron B, aluminum Al, gallium Ga, indium In, silicon Si, germanium Ge, tin Sn, lead Pb, bismuth Bi, cadmium Cd, silver Ag, zinc Zn, hafnium Hf, zirconium Zr, Yttrium Y, palladium Pd and platinum Pt. These may be in crystalline or amorphous form.

In addition, examples of the alloys or compounds of these metal elements or semi-metal elements include compounds expressed by the chemical formula MasMbtLiu, or MapMcqMdr. In these chemical formulae, Ma represents at least one kind selected from metal elements and semi-metal elements capable of forming alloys with lithium Li; Mb represents at least one kind selected from metal elements and semi-metal elements other than Ma; Mc represents at least one kind selected from semi-metal elements; and Md represents at least one kind selected from metal elements and semi-metal elements other than Md and Ma. Additionally, the values of s, t, u, p, q and r satisfy the conditions that s>0, t≧0, u≧0, p>0, q>0, and r≧0.

Of these, this anode material is preferably a simple substance, alloy or compound of the metal elements or semi-metal elements in the group 4B in the short-form periodic table, particularly preferably a simple substance of silicon Si or tin Sn or an alloy or compound thereof. This is because a simple substance, alloy and compound of silicon Si or tin Sn have large capacities of occlusion and discharge of lithium Li, and can make high the energy density of an anode 22 as compared with usual graphite depending on combination.

Specific examples of such alloys or compounds include SiB4, SiB6, Mg2Si, Ni2Si, TiSi2, MoSi2, CoSi2, NiSi2, CaSi2, CrSi2, Cu5Si, FeSi2, MnSi2, NbSi2, TaSi2, VSi2, WSi2, ZnSi2, SiC, Si3N4, Si2N20, SiOv (0<v≦2), SnOw (0<w≦2), SnSiO3, LiSiO, LiSnO, Mg2Sn, and alloys containing tin Sn and cobalt Co.

Of these, this anode material preferably is a CoSnC-containing material containing as constituent elements tin Sn, cobalt Co and carbon C, in which the content of carbon is 9.9 mass % or more and 29.7 mass % or less, and the ratio (Co/(Sn+Co)) of cobalt Co to the total of the tin Sn and the cobalt Co (by mass %) is 30 mass % or more and 70 mass % or less. This is because these composition ranges can provide a high energy density and also excellent cycle characteristics.

This CoSnC-containing material may further contain other constituent elements, as necessary. The other constituent elements preferably include, for example, silicon Si, iron Fe, nickel Ni, chromium Cr, indium In, niobium Nb, germanium Ge, titanium Ti, molybdenum Mo, aluminum Al, phosphorus P, gallium Ga or bismuth Bi, and may include two or more kinds thereof. This is because the capacity or the cyclic characteristics can be improved.

It is preferable that the CoSnC-containing material has a phase containing tin Sn, cobalt Co and carbon C, and that this phase has a low crystalline or amorphous structure. Further, in the CoSnC-containing material, at least part of the carbon as the constituent element is preferably bonded to a metal element or semi-metal element that is another constituent element. This is because a decrease in cycle characteristics is thought to be due to aggregation or crystallization of tin Sn or the like, but bonding of the carbon to another element can suppress such aggregation or crystallization.

An example of the measurement method of investigating a bonding state of an element is X-ray Photoelectron Spectroscopy (XPS). In XPS, the peak of the 1s orbital (C1s) of carbon in graphite appears at 284.5 eV when using an apparatus in which the peak of the 4f orbital (Au4f) of a gold atom is energy calibrated so as to be obtained at 84.0 eV. Alternatively, the peak of a surface contaminated carbon appears at 284.8 eV. On the other hand, when the charge density of a carbon element becomes high, for example, when carbon is bonded to a metal element or semi-metal element, the peak of C1s appears in a region lower than 284.5 eV. In other words, in the case where the peak of a combination wave of C1s obtained from a CoSnC-containing material appears in a region lower than 284.5 eV, at least part of the carbon atoms contained in the CoSnC-containing material is linked to a metal element or semi-metal element that is another constituent element.

In the XPS measurement, for example, the C1s peak is used for correction of the spectral energy axis. Usually, because of the presence of the surface contaminated carbon on the surface, the C1s peak of the surface contaminated carbon is set to be 284.8 eV, which is used as an energy reference. In the XPS measurement, the wave shape of the C1s peak is obtained as a shape including the surface contaminated carbon peak and the carbon peak in the CoSnC-containing material. Thus, for example, the surface contaminated carbon peak is made separated from the carbon peak in the CoSnC-containing material by analysis using commercially available software. In the analysis of a wave shape, the position of the primary peak present on the lowest binding energy side is set to be an energy reference (284.8 eV).

The anode active material 4B may be formed by any of, for example, a gas phase method, a liquid phase method, a baking method, and a coating method, or by a combination of two or more methods thereof. The baking method is a method of mixing a particulate anode active material with a binding agent or a solvent, etc. for molding and then, heat treating the resulting material at a temperature higher than a melting point of the binding agent, for example.

The formation by a gas phase method, a liquid phase method or a baking method is preferred because the anode active material layer 4B and the anode current collector 4A may be alloyed at least a part of an interface therebetween during formation. Further, they may be alloyed by heat treatment under vacuum or nonoxidative atmosphere. Specifically, at the interface, preferably, the constituent elements of the anode current collector 4A diffuse into the anode active material layer 4B, or the constituent elements of the anode active material diffuse into the anode current collector 4A, or they diffuse into each other. This is because it the destruction of the anode active material layer 4B by expansion and contraction along with charge and discharge can be suppressed and also electron conductivity between the anode active material layer 4B and the anode current collector 4A can be improved.

As the gas phase method available are, for example, a physical deposition method or a chemical deposition method. Specifically, usable is a vacuum deposition method, a sputtering method, an ion plating method, a laser abrasion method, a thermal chemical vapor deposition (CVD) method, a plasma CVD method or the like. As the liquid phase method, a well-known technique such as electrolyte gold plating or non-aqueous electrolyte gold plating can be employed. The baking method also may be a well-known technique, and usable examples thereof include atmospheric baking, reaction baking and hot plate baking. In the case of coating, the anode can also be applied as in the cathode 2.

The outer covering cup 5 is a container made from an electroconductive metal to house the anode 4, and is an external anode. Specifically, as the outer covering cup 5 used is, for example, a metal container made from aluminum Al, stainless SUS, or iron having nickel Ni plated on its surface.

The separator 3 separates the cathode 2 from the anode 4, and allows lithium ions to pass in a non-aqueous electrolyte solution while preventing a short circuit of a current due to contact of both the electrodes. The separator 3 is formed from a fine porous film having many fine pores. Here, the fine porous film refers to a resin film having many pores having an average pore diameter of about 5 μm or more. Additionally, the material of the separator 3 may be materials that have been used for batteries in the past. Of these, fine porous films made from polypropylene, polyolefin, or the like are used that are excellent in a short circuit prevention effect and capable of safety improvement of a battery caused by a shut-down effect.

The gasket 7 is configured to be incorporated into the outer covering cup 5 and integrated, and is formed with, for example, an organic resin such as polypropylene. The gasket 7 insulates the outer covering can 6 serving as an external cathode from the outer covering cup 5 serving as an external anode, and also functions to prevent the leakage of the non-aqueous electrolyte solution loaded into the outer covering cup 5 and the outer covering can 6.

Next, a method of the first example for manufacturing a non-aqueous electrolyte battery will be described. The cathode 2 is fabricated, for example, as described below. First, for example, a cathode active material, an electroconductive agent and a binding agent are dispersed in a non-aqueous solvent or the like to prepare a cathode mixture application liquid. Then, the cathode mixture application liquid is uniformly applied, for example, to the metal foil-like cathode collector 2A such as an aluminum Al foil and dried, followed by being compression molded, to form the cathode active material layer 2B. This provides the cathode 2.

The anode 4 is fabricated, for example, as described below. First, for example, an anode active material and a binding agent are dispersed in a non-aqueous solvent or the like to prepare an anode mixture application liquid. The anode mixture application liquid is uniformly applied, for example, to the metal foil-like anode current collector 4A such as a copper Cu foil and dried, followed by being compressed, to form the anode active material layer 4B. This provides the anode 4.

Next, the cathode 2 is accommodated in the outer covering can 6, the anode 4 is housed in the outer covering cup 5, and the separator 3 formed from a polypropylene porous film or the like is placed between the cathode 2 and the anode 4. With this, the non-aqueous electrolyte battery has an internal structure in which the cathode 2, the separator 3 and the anode 4 are laminated in this order.

Then, the non-aqueous electrolyte solution is poured into the outer covering can 6 and the outer covering cup 5 to fix the outer covering can 6 and the outer covering cup 5 via the gasket 7. As described above, the non-aqueous electrolyte battery is obtained.

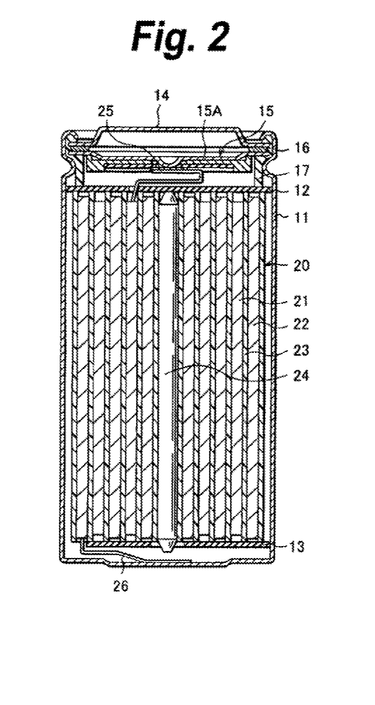

Next, a second example of a non-aqueous electrolyte battery using the non-aqueous electrolyte according to one embodiment of the invention will be set forth. FIG. 2 is a cross sectional view showing a configuration of the second example of the non-aqueous electrolyte battery.

This non-aqueous electrolyte battery is, for example, a non-aqueous electrolyte secondary battery, e.g., a lithium ion secondary battery. The non-aqueous electrolyte battery is referred to as a so-called cylindrical type. That is, a winded electrode member 20 produced by winding a pair of a band-shaped cathode 21 and a band-shaped anode 22 via a separator 23 is located in the inside of a substantially hollow cylindrical battery can 11. The separator 23 is impregnated with a non-aqueous electrolyte solution that is a liquid non-aqueous electrolyte. The battery can 11 is constituted by, for example, iron Fe plated with nickel Ni, and its one end is closed and the other end is open. In the inside of the battery can 11, a pair of an insulator plate 12 and an insulator plate 13 are each placed orthogonally to a winded periphery so as to pinch the winded electrode member 20 therebetween.

At the open end of the battery can 11, a battery lid 14 and a safety valve mechanism 15 and a positive temperature coefficient (PTC) element 16 arranged inside the battery lid 14 are fixed by caulking via the gasket 17, and the inside of the battery can 11 is enclosed.

The battery lid 14 is made of, for example, a material similar to that of the battery can 11. The safety valve mechanism 15 is electrically connected to the battery lid 14 via the positive temperature coefficient element 16, and when the inner pressure of the cell reaches a certain amount or more due to, for example, inside short circuit or heating from outside, a disk plate 15A is inverted to cut the electrical connection between the battery lid 14 and the winded electrode member 20. The positive temperature coefficient element 16 restricts the current by increasing the resistance value when the temperature is raised, in order to prevent abnormal heat generation due to a large current. The gasket 17 is made of, for example, an insulating material and its surface is coated with asphalt.

The winded electrode member 20 is winded, for example, around a center pin 24. A cathode lead 25 made of aluminum Al or the like is connected to the cathode 21 of the winded electrode member 20, and an anode lead 26 made of nickel or the like is connected to the anode 22. The cathode lead 25 is welded to the safety valve mechanism 15 to thereby be electrically connected to the battery lid 14, and the anode lead 26 is electrically connected with the battery can 11 by welding.

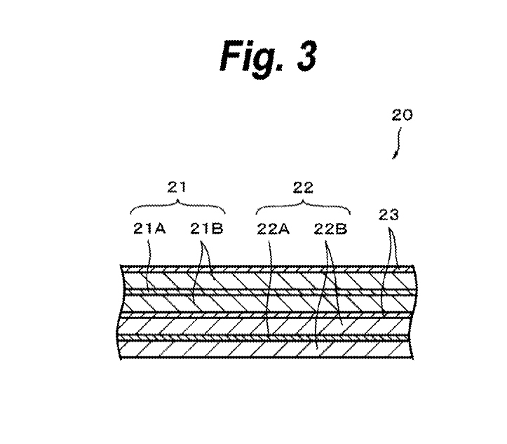

FIG. 3 is a cross sectional view showing a part enlarged of the winded electrode member 20 shown in FIG. 2. Referring to FIG. 3, the cathode 21, the anode 22 and the separator 23 constituting the non-aqueous electrolyte battery will be described one by one hereinafter.

The cathode 21 has, for example, a structure in which cathode active material layers 21B are arranged on both the faces of a cathode collector 21A having a pair of faces opposite to each other. Although not shown, the winded electrode member may have a region in which the cathode active material layer 21B is provided only on one face of the cathode collector 21A. The cathode collector 21A is constituted, for example, by a metal foil such as an aluminum foil.

The cathode active material layer 21B is configured to include, for example, a cathode material capable of occlusion and discharge of lithium Li, and is configured to include as necessary an electroconductive agent such as graphite and a binding agent such as poly(vinylidene fluoride) (PVDE). Since the cathode material capable of occlusion and discharge of lithium Li is similar to the material as described in the above-described first example, detailed description will be omitted.

The anode 22 has, for example, a structure in which anode active material layers 22B are arranged on both the faces of an anode collector 22A having a pair of faces opposite to each other. Although not shown, the winded electrode member may have a region in which the anode active material layer 22B is provided only on one face of the anode collector 22A. The anode collector 22A is constituted, for example, by a metal foil such as a copper foil.

The anode active material layer 22B is configured to include, for example, an anode material capable of occlusion and discharge of lithium Li, and is configured to include as necessary a binding agent similar to that of the cathode active material layer 21B. Since the anode material capable of occlusion and discharge of lithium Li is similar to the material as described in the above-described first example, detailed description will be omitted.

The separator 23 is similar to the separator 3 described in the above-described first example, and thus, detailed description will be omitted.

Next, one example of the method of the second example for manufacturing the non-aqueous electrolyte battery will be described. First, for example, a cathode active material, an electroconductive agent, and a binding agent are admixed to prepare a cathode mixture, and the cathode mixture is dispersed in a solvent such as N-methyl-2-pyrolidone to produce a paste-like cathode mixture slurry. Next, this cathode mixture slurry is applied to the cathode collector 21A and the solvent is dried, and then compression molded by means of a roll press machine or the like to form the cathode active material layer 21B. This provides the cathode 21.

In addition, for example, an anode active material and a binding agent are admixed to prepare an anode mixture, and this anode mixture is dispersed in a solvent such as N-methyl-2-pyrolidone to produce a paste-like anode mixture slurry. Next, this anode mixture slurry is applied to the anode collector 22A and the solvent is dried, and then compression molded by means of a roll press machine or the like to form the anode active material layer 22B. This provides the anode 22.

Next, the cathode lead 25 is fixed to the cathode collector 21A by welding or the like, and also the anode lead 26 is fixed to the anode collector 22A by welding or the like. Thereafter, the cathode 21 and the anode 22 are winded via the separator 23, the leading end of the cathode lead 25 is welded to the safety valve mechanism 15 and also the leading end of the anode lead 26 is welded to the battery can 11, so that the winded cathode 21 and the anode 22 are pinched with the pair of insulator plates 12, 13 so as to be housed in the inside of the battery can 11. The cathode 21 and the anode 22 are housed within the battery can 11 and then a non-aqueous electrolyte solution is poured into the inside of the battery can 11 and impregnated into the separator 23. Then, to the open end of the battery can 11, the battery lid 14, the safety valve mechanism 15 and the positive temperature coefficient element 16 are fixed by caulking via the gasket 17. This provides the non-aqueous electrolyte battery shown in FIG. 2.

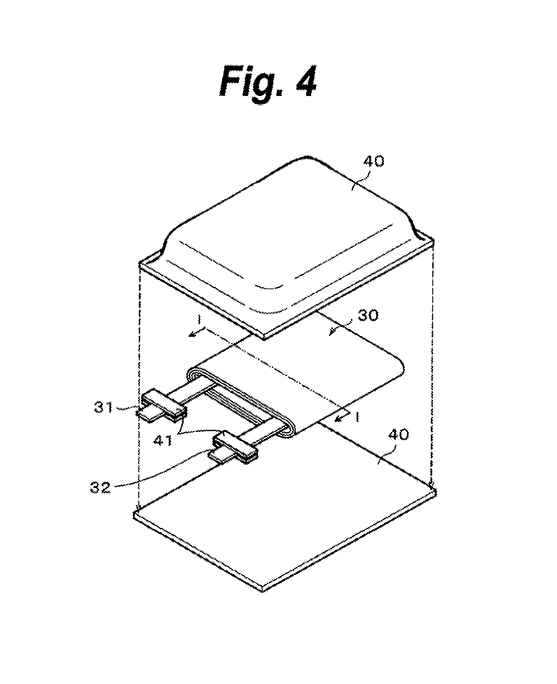

Then, a third example of the non-aqueous electrolyte battery will be described. FIG. 4 is a cross sectional view showing one exemplary configuration of the third example of the non-aqueous electrolyte battery. This non-aqueous electrolyte battery is configured such that a winded electrode member 30 provided with a cathode lead 31 and an anode lead 32 is housed in the inside of a film-like outer covering member 40, so that the non-aqueous electrolyte battery can be made miniaturized, lightened and thinned.

The cathode lead 31 and the anode lead 32 are each derived from the inside of the outer covering member 40 outward, for example, in a same direction. The cathode lead 31 and the anode lead 32 are each configured by, for example, a metal material such as aluminum Al, copper Cu, nickel Ni or stainless SUS, each being in a thin plate or network form.

The outer covering member 40 is configured by, for example, a rectangular aluminum laminate film produced by laminating a nylon film, an aluminum foil and a polyethylene film in this order. The outer covering member 40 has, for example, the polyethylene film side and the winded electrode member 30 arranged such that they face each other, and outer edges are affixed to each other by fusion or an adhesive. An adhesion film 41 for preventing the invasion of external air is inserted between the outer covering members 40 and the cathode lead 31 and the anode lead 32. The adhesion film 41 is made of a material having adhesion properties to the cathode lead 31 and the anode lead 32, for example, a polyolefin resin such as polyethylene, polypropylene, modified polyethylene or modified polypropylene.

Additionally, the outer covering member 40 may be made of, in place of the above-mentioned aluminum laminate film, a polymer film or a metal film such as a laminate film or polypropylene having another structure.

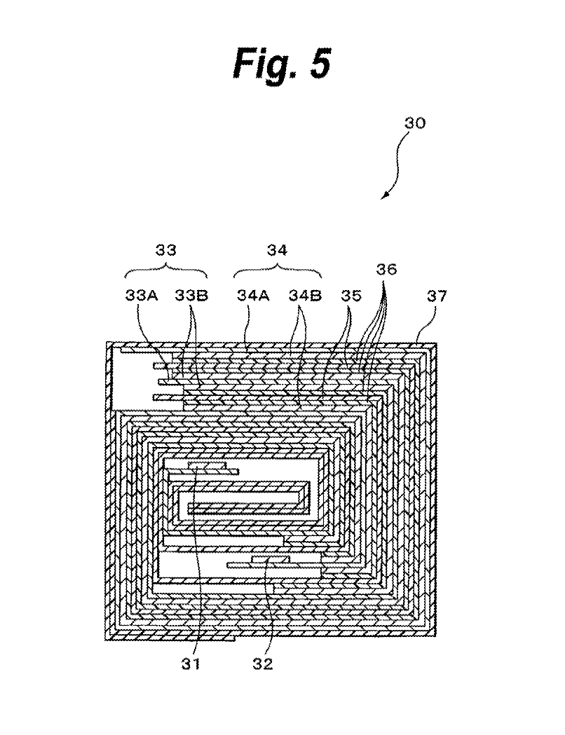

FIG. 5 is a cross sectional view taken along the line I-I of the winded electrode member 30 shown in FIG. 3. The winded electrode member 30 is produced by laminating and winding a cathode 33 and an anode 34 via a separator 35 and an electrolyte layer 36, and the outermost periphery is protected by a protection tape 37.

The cathode 33 has a structure in which cathode an active material layer(s) 33B is/are provided on one face or both the faces of a cathode collector 33A. The anode 34 has a structure in which an anode active material layer(s) 34B is/are provided on one face or both the faces of an anode collector 34A, and the anode active material layer 34B is arranged so as to face the cathode active material layer 33B. The configurations of the cathode collector 33A, the cathode active material layer 33B, the anode collector 34A, the anode active material layer 34B and the separator 35 are respectively similar to those of the cathode collector 21A, the cathode active material layer 21B, the anode collector 22A, the anode active material layer 22B and the separator 23 described in the first and second examples. Thus, detailed description will be omitted.

The electrolyte layer 36 contains the above-described non-aqueous electrolyte solution and a polymer compound serving as a holder holding the non-aqueous electrolyte solution, and is a so-called gel-like non-aqueous electrolyte. The gel-like electrolyte layer 36 is preferable because it can obtain a high ionic conductivity and also can prevent the leakage of the solution from the battery. In addition, the electrolyte layer 36 is not made to render a polymer compound to hold a non-aqueous electrolyte solution and may directly use a non-aqueous electrolyte solution.

Next, one example of the method of the third example for manufacturing a battery will be described. First, a precursor solution containing a solvent, an electrolyte salt, a polymer compound and a mixture solvent is applied to each of the cathode 33 and the anode 34, and the mixture solution is made to vaporize to form the electrolyte layer 36. Thereafter, the cathode lead 31 is attached by welding to the end of the cathode collector 33A and also the anode lead 32 is attached by welding to the end of the anode collector 34A.

Then, the cathode 33 and the anode, having the electrolyte layer 36 formed therein, are laminated via the separator 35 to make a laminate, and then this laminate is winded in its longitudinal direction and the protection tape 37 is affixed onto the outermost periphery to form the winded electrode member 30. Finally, for example, the winded electrode member 30 is inserted between the outer covering members 40 and the outer edges of the outer covering members 40 are affixed to each other by heat fusion or the like for enclosure purposes. During the time, the adhesion film 41 is inserted between the cathode lead 31 and the anode lead 32 and the outer covering members 40. This provides the secondary battery indicated in FIG. 4.

This secondary battery may be also fabricated in the following. First, the cathode 33 and the anode 34 are fabricated as described above, and the cathode lead 31 and anode lead 32 are fixed to the cathode 33 and the anode 34. Then, the cathode 33 and the anode 34 are laminated via the separator and winded, and the protection tape 37 is affixed onto the outermost periphery to form the winded electrode member 30. Next, this winded electrode member 30 is pinched between the outer covering members 40 and the outer peripheral edges excluding one side are heat fused to make a bag shape, whereby the winded electrode member is housed in the outer peripheral member 40. Subsequently, a composition for the electrolyte is prepared that contains a solvent, an electrolyte salt, a compound represented by Chemical formula 3, a monomer that is a raw material of a polymer compound, a polymerization initiator and as necessary another material such as a polymerization inhibitor, and the composition is poured into the inside of the outer covering member 40.

After pouring the composition for the electrolyte, the opening of the outer covering member 40 is heat fused under a vacuum atmosphere for sealing. Next, heat is applied to polymerize the monomer to make a polymer compound, thereby forming the electrolyte layer 36. As the result, the secondary battery shown in FIG. 4 is obtained.

### EXAMPLE

Specific examples will be described with reference to FIG. 1 according to an embodiment. However, this invention is by no means limited to these examples only. Herein, in the descriptions below, Compounds 1 to 15 refer to the compounds represented by (7-1) to (7-15) of Chemical formula 7, respectively. In addition, compounds commercially available were used as Compounds 1 to 5 and 11. Compounds 6, 8 to 10 and 12 to 14 were synthesized using corresponding oxocarbonic acids and alcohols as starting materials according to the methods described in “Tetrahedron vol. 56, pp. 9325 to 9333, 2001”. Compound 7 was synthesized from squaric acid and N,O-bis(trimethylsilyl)acetamide according to the method described in “Journal of American Chemical Society, vol. 98, pp. 3641 to 3644, 1976.” Compound 15 was synthesized from squaric acid and N,O-bis(tert-butylmethylsilyl)acetamide by a method similar to the case of Compound 7.

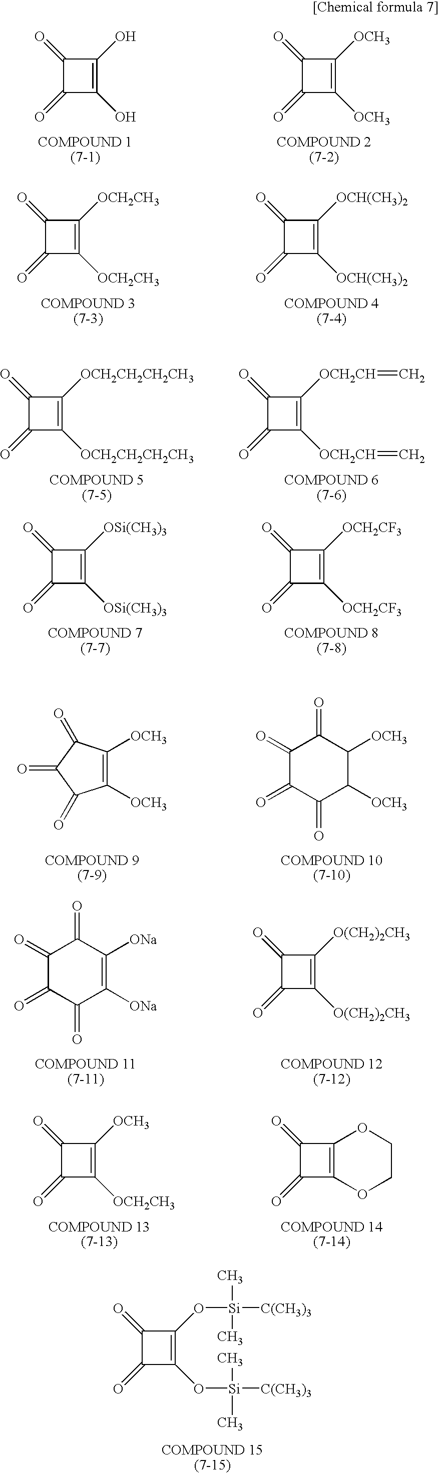

### Example 1-1

A coin-shaped secondary battery shown in FIG. 1 was fabricated as a non-aqueous electrolyte secondary battery of Example 1-1. In this secondary battery, a cathode 2 and an anode 4 were laminated via a separator 3 made of a fine porous polypropylene film impregnated with an electrolyte solution, and the resulting laminate was sandwiched between an outer covering can 6 and an outer covering cup 5 and then was fixed by caulking via a gasket 7.

First, 94 mass parts of a lithium cobalt complex oxide (LiCoO2) as a cathode active material, 3 mass parts of graphite as an electroconductive agent, and 3 mass parts of poly(vinylidene fluoride) as a binding agent were mixed and then N-methyl-2-pyrolidone was added to the mixture to obtain a cathode mixture slurry.

Next, the resultant cathode mixture slurry was uniformly applied to a cathode collector 2A constituted by a 20 μm thick aluminum foil and dried to form a 70 μm thick cathode active material layer 2B. Then, the cathode collector 2A having the cathode active material layer 2B formed thereon was punched to a circular shape with a diameter of 15 mm, thereby obtaining the cathode 2.

Further, 97 mass parts of graphite as an anode active material and 3 mass parts of poly(vinylidene fluoride) as a binding agent were mixed and N-methyl-2-pyrolidone was added to the mixture. The resulting mixture was uniformly applied to an anode collector 4A constituted by a copper foil having a thickness of 15 μm and dried to form a 70 μm thick anode active material layer 4B. Thereafter, the anode collector 4A having the anode active material layer 4B formed thereon was punched to a circular shape with a diameter of 15 mm, thereby obtaining the anode 4.

Next, the cathode 2 and the anode 4 were laminated via the separator made of a fine porous polypropylene film with a thickness of 25 μm and an electrolyte solution was poured into the separator 3. Then, the resulting material was placed in the outer covering cup 5 and the outer covering can 6 which were made of stainless steel, and these are fixed by caulking to obtain the secondary battery of Example 1-1. As the electrolyte solution, used was an electrolyte solution prepared by dissolving LiPF6 as an electrolyte salt in a solvent prepared by mixing of ethylene carbonate (EC) with diethylene carbonate (DEC) in a weight ratio of 3:7 (EC:DEC) such that the electrolyte salt was 1.0 mol/kg and adding Compound 1 thereto such that compound 1 was at 1 wt %.

### Example 1-2

A secondary battery of Example 1-2 was fabricated in the same manner as in Example 1-1 except that Compound 2 was added at 1 wt % instead of addition of Compound 1.

### Example 1-3

A secondary battery of Example 1-3 was fabricated in the same manner as in Example 1-1 except that Compound 3 was added at 1 wt % instead of addition of Compound 1.

### Example 1-4

A secondary battery of Example 1-4 was fabricated in the same manner as in Example 1-1 except that Compound 4 was added at 1 wt % instead of addition of Compound 1.

### Example 1-5

A secondary battery of Example 1-5 was fabricated in the same manner as in Example 1-1 except that Compound 5 was added at 1 wt % instead of addition of Compound 1.

### Example 1-6

A secondary battery of Example 1-6 was fabricated in the same manner as in Example 1-1 except that Compound 6 was added at 1 wt % instead of addition of Compound 1.

### Example 1-7

A secondary battery of Example 1-7 was fabricated in the same manner as in Example 1-1 except that Compound 7 was added at 1 wt % instead of addition of Compound 1.

### Example 1-8

A secondary battery of Example 1-8 was fabricated in the same manner as in Example 1-1 except that Compound 8 was added at 1 wt % instead of addition of Compound 1.

### Example 1-9

A secondary battery of Example 1-9 was fabricated in the same manner as in Example 1-1 except that Compound 9 was added at 1 wt % instead of addition of Compound 1.

### Example 1-10

A secondary battery of Example 1-10 was fabricated in the same manner as in Example 1-1 except that Compound 10 was added at 1 wt % instead of addition of Compound 1.

### Example 1-11

A secondary battery of Example 1-11 was fabricated in the same manner as in Example 1-1 except that Compound 12 was added at 1 wt % instead of addition of Compound 1.

### Example 1-12

A secondary battery of Example 1-12 was fabricated in the same manner as in Example 1-1 except that Compound 13 was added at 1 wt % instead of addition of Compound 1.

### Example 1-13

A secondary battery of Example 1-13 was fabricated in the same manner as in Example 1-1 except that Compound 14 was added at 1 wt % instead of addition of Compound 1.

### Example 1-14

A secondary battery of Example 1-14 was fabricated in the same manner as in Example 1-1 except that Compound 15 was added at 1 wt % instead of addition of Compound 1.

### Example 1-15

A secondary battery of Example 1-15 was fabricated in the same manner as in Example 1-2 except that an electrolyte solution obtained by further addition of vinylene carbonate (VC) at 1 wt % was used.

### Example 1-16

A secondary battery of Example 1-16 was fabricated in the same manner as in Example 1-2 except that an electrolyte solution obtained by further addition of 4-fluoro-1,3-dioxolane-2-one (FEC) at 1 wt % was used.

### Example 1-17

A secondary battery of Example 1-17 was fabricated in the same manner as in Example 1-2 except that an electrolyte solution obtained by further addition of 4,5-difluoro-1,3-dioxolane-2-one (DFEC) at 1 wt % was used.

### Example 1-18

A secondary battery of Example 1-18 was fabricated in the same manner as in Example 1-2 except that an electrolyte solution obtained by further addition of propene sultone (PRS) at 1 wt % was used.

### Example 1-19

A secondary battery of Example 1-19 was fabricated in the same manner as in Example 1-2 except that an electrolyte solution obtained by further addition of succinic anhydride at 1 wt % was used.

### Example 1-20

A secondary battery of Example 1-20 was fabricated in the same manner as in Example 1-2 except that an electrolyte solution obtained by further addition of 2-sulfobenzoic anhydride at 1 wt % was used.

### Example 1-21

A secondary battery of Example 1-21 was fabricated in the same manner as in Example 1-2 except that an electrolyte solution obtained by further addition of ethylene sulfite at 1 wt % was used.

### Example 1-22

A secondary battery of Example 1-22 was fabricated in the same manner as in Example 1-2 except that LiPF6 and LiBF4 as electrolyte salts were dissolved so as to be 0.9 mol/kg and 0.1 mol/kg, respectively.

### Example 1-23

A secondary battery of Example 1-23 was fabricated in the same manner as in Example 1-2 except that LiPF6 and lithium bisoxalate borate (LiBOB) as electrolyte salts were dissolved so as to be 0.9 mol/kg and 0.1 mol/kg, respectively.

### Example 1-24

A secondary battery of Example 1-24 was fabricated in the same manner as in Example 1-2 except that LiPF6 and lithium bis(trifluoromethanesulfonyl)imide (LiTFSI) as electrolyte salts were dissolved so as to be 0.9 mol/kg and 0.1 mol/kg, respectively.

### Example 1-25

A secondary battery of Example 1-25 was fabricated in the same manner as in Example 1-2 except that LiPF6 and lithium 1,3-perfluoropropanedisulfonylimide as electrolyte salts were dissolved so as to be 0.9 mol/kg and 0.1 mol/kg, respectively.

### Comparison 1-1

A secondary battery of Comparison 1-1 was fabricated in the same manner as in Example 1-1 except that Compound 1 was not added.

### Comparison 1-2

A secondary battery of Comparison 1-2 was fabricated in the same manner as in Example 1-1 except that Compound 11 was added at 1 wt % instead of addition of Compound 1.

### Example 2-1

A secondary battery of Example 2-1 was fabricated in the same manner as in Example 1-1 except that an anode 4 fabricated as described below was used. An anode active material layer 4B with a thickness of 5 μm constituted by silicon Si was formed on an anode collector 4A constituted by a 15 μm thick copper foil by vapor deposition. Thereafter, the anode collector 4A having the anode active material layer 4B formed thereon was punched to a circular shape with a diameter of 16 mm. This provided the anode 4.

### Example 2-2

A secondary battery of Example 2-2 was fabricated in the same manner as in Example 2-1 except that Compound 2 was added at 1 wt % instead of addition of Compound 1.

### Example 2-3

A secondary battery of Example 2-3 was fabricated in the same manner as in Example 2-1 except that Compound 3 was added at 1 wt % instead of addition of Compound 1.

### Example 2-4

A secondary battery of Example 2-4 was fabricated in the same manner as in Example 2-1 except that Compound 3 was added at 5 wt % instead of addition of Compound 1.

### Example 2-5

A secondary battery of Example 2-5 was fabricated in the same manner as in Example 2-1 except that Compound 4 was added at 1 wt % instead of addition of Compound 1.

### Example 2-6

A secondary battery of Example 2-6 was fabricated in the same manner as in Example 2-1 except that Compound 5 was added at 1 wt % instead of addition of Compound 1.

### Example 2-7

A secondary battery of Example 2-7 was fabricated in the same manner as in Example 2-1 except that Compound 5 was added at 5 wt % instead of addition of Compound 1.

### Example 2-8

A secondary battery of Example 2-8 was fabricated in the same manner as in Example 2-1 except that Compound 6 was added at 1 wt % instead of addition of Compound 1.

### Example 2-9

A secondary battery of Example 2-9 was fabricated in the same manner as in Example 2-1 except that Compound 6 was added at 5 wt % instead of addition of Compound 1.

### Example 2-10

A secondary battery of Example 2-10 was fabricated in the same manner as in Example 2-1 except that Compound 7 was added at 1 wt % instead of addition of Compound 1.

### Example 2-11

A secondary battery of Example 2-11 was fabricated in the same manner as in Example 2-1 except that Compound 7 was added at 5 wt % instead of addition of Compound 1.

### Example 2-12

A secondary battery of Example 2-12 was fabricated in the same manner as in Example 2-1 except that Compound 8 was added at 1 wt % instead of addition of Compound 1.

### Example 2-13

A secondary battery of Example 2-13 was fabricated in the same manner as in Example 2-1 except that Compound 9 was added at 1 wt % instead of addition of Compound 1.

### Example 2-14

A secondary battery of Example 2-14 was fabricated in the same manner as in Example 2-1 except that Compound 10 was added at 1 wt % instead of addition of Compound 1.

### Example 2-15

A secondary battery of Example 2-15 was fabricated in the same manner as in Example 2-1 except that Compound 12 was added at 1 wt % instead of addition of Compound 1.

### Example 2-16

A secondary battery of Example 2-16 was fabricated in the same manner as in Example 2-1 except that Compound 13 was added at 1 wt % instead of addition of Compound 1.

### Example 2-17

A secondary battery of Example 2-17 was fabricated in the same manner as in Example 2-1 except that Compound 14 was added at 1 wt % instead of addition of Compound 1.

### Example 2-18

A secondary battery of Example 2-18 was fabricated in the same manner as in Example 2-1 except that Compound 15 was added at 1 wt % instead of addition of Compound 1.

### Example 2-19

A secondary battery of Example 2-19 was fabricated in the same manner as in Example 2-2 except that an electrolyte solution was used that was prepared by dissolving LiPF6 as an electrolyte salt in a solvent prepared by mixing 4-fluoro-1,3-dioxolane-2-one (FEC) with diethylene carbonate (DEC) in a weight ratio of 50:50 (FEC:DEC) such that the electrolyte salt was 1.0 mol/kg and adding Compound 2 to the mixture such that Compound 2 was at 1 wt %.

### Example 2-20

A secondary battery of Example 2-20 was fabricated in the same manner as in Example 2-19 except that Compound 6 was added at 1 wt % instead of addition of Compound 2.

### Example 2-21

A secondary battery of Example 2-21 was fabricated in the same manner as in Example 2-19 except that Compound 7 was added at 1 wt % instead of addition of Compound 2.

### Example 2-22

A secondary battery of Example 2-22 was fabricated in the same manner as in Example 2-19 except that Compound 8 was added at 1 wt % instead of addition of Compound 2.

### Example 2-23

A secondary battery of Example 2-23 was fabricated in the same manner as in Example 2-2 except that an electrolyte solution was used that was prepared by dissolving LiPF6 as an electrolyte salt in a solvent prepared by mixing propylene carbonate (PC), 4,5-difluoro-1,3-dioxolane-2-one (DFEC) and diethylene carbonate (DEC) in a weight ratio of 40:10:50 (PC:DFEC:DEC) such that the electrolyte salt was 1.0 mol/kg and adding Compound 2 to the mixture such that Compound 2 was at 1 wt %.

### Example 2-24

A secondary battery of Example 2-24 was fabricated in the same manner as in Example 2-23 except that Compound 6 was added at 1 wt % instead of addition of Compound 2.

### Example 2-25

A secondary battery of Example 2-25 was fabricated in the same manner as in Example 2-23 except that Compound 7 was added at 1 wt % instead of addition of Compound 2.

### Example 2-26

A secondary battery of Example 2-26 was fabricated in the same manner as in Example 2-23 except that Compound 8 was added at 1 wt % instead of addition of Compound 2.

### Example 2-27

A secondary battery of Example 2-27 was fabricated in the same manner as in Example 2-2 except that an electrolyte solution was used that was prepared by dissolving LiPF6 as an electrolyte salt in a solvent prepared by mixing propylene carbonate (PC), 4-fluoro-1,3-dioxolane-2-one (FEC), 4,5-difluoro-1,3-dioxolane-2-one (DFEC) and diethylene carbonate (DEC) in a weight ratio of 30:10:10:50 (PC:FEC:DFEC:DEC) such that the electrolyte salt was 1.0 mol/kg and adding Compound 2 to the mixture such that Compound 2 was at 1 wt %.

### Example 2-28

A secondary battery of Example 2-28 was fabricated in the same manner as in Example 2-27 except that Compound 6 was added at 1 wt % instead of addition of Compound 2.

### Example 2-29

A secondary battery of Example 2-29 was fabricated in the same manner as in Example 2-27 except that Compound 7 was added at 1 wt % instead of addition of Compound 2.

### Example 2-30

A secondary battery of Example 2-30 was fabricated in the same manner as in Example 2-27 except that Compound 8 was added at 1 wt % instead of addition of Compound 2.

### Example 2-31

A secondary battery of Example 2-31 was fabricated in the same manner as in Example 2-2 except that an electrolyte solution obtained by further addition of vinylene carbonate (VC) at 1 wt % was used.

### Example 2-32

A secondary battery of Example 2-32 was fabricated in the same manner as in Example 2-2 except that an electrolyte solution obtained by further addition of propene sultone (PRS) at 1 wt % was used.

### Example 2-33

A secondary battery of Example 2-33 was fabricated in the same manner as in Example 2-2 except that an electrolyte solution obtained by further addition of succinic anhydride at 1 wt % was used.

### Example 2-34

A secondary battery of Example 2-34 was fabricated in the same manner as in Example 2-2 except that an electrolyte solution obtained by further addition of 2-sulfobenzoic anhydride at 1 wt % was used.

### Example 2-35

A secondary battery of Example 2-35 was fabricated in the same manner as in Example 2-2 except that an electrolyte solution obtained by further addition of ethylene sulfite at 1 wt % was used.

### Example 2-36

A secondary battery of Example 2-36 was fabricated in the same manner as in Example 2-2 except that LiPF6 and LiBF4 as electrolyte salts were dissolved so as to be 0.9 mol/kg and 0.1 mol/kg, respectively.

### Example 2-37

A secondary battery of Example 2-37 was fabricated in the same manner as in Example 2-2 except that LiPF6 and lithium bisoxalate borate (LiBOB) as electrolyte salts were dissolved so as to be 0.9 mol/kg and 0.1 mol/kg, respectively.

### Example 2-38

A secondary battery of Example 2-38 was fabricated in the same manner as in Example 2-2 except that LiPF6 and lithium bis(trifluoromethanesulfonyl)imide (LiTFSI) as electrolyte salts were dissolved so as to be 0.9 mol/kg and 0.1 mol/kg, respectively.

### Example 2-39

A secondary battery of Example 2-39 was fabricated in the same manner as in Example 2-2 except that LiPF6 and lithium 1,3-perfluoropropanedisulfonylimide as electrolyte salts were dissolved so as to be 0.9 mol/kg and 0.1 mol/kg, respectively.

### Comparison 2-1

A secondary battery of Comparison 2-1 was fabricated in the same manner as in Example 2-1 except that Compound 1 was not added.

### Comparison 2-2

A secondary battery of Comparison 2-2 was fabricated in the same manner as in Example 2-1 except that Compound 11 was added at 1 wt % instead of addition of Compound 1.

### Comparison 2-3

A secondary battery of Comparison 2-3 was fabricated in the same manner as in Example 2-19 except that Compound 2 was not added.

### Comparison 2-4

A secondary battery of Comparison 2-4 was fabricated in the same manner as in Example 2-23 except that Compound 2 was not added.

### Comparison 2-5

A secondary battery of Comparison 2-5 was fabricated in the same manner as in Example 2-27 except that Compound 2 was not added.

### Example 3-1

A secondary battery of Example 3-1 was fabricated in the same manner as in Example 1-1 except that an anode 4 was fabricated as described below. When the anode 4 was fabricated, first, a tin/cobalt/indium/titanium alloy powder was mixed with a carbon powder, and then a CoSnC-containing material was synthesized making use of a mechanochemical reaction. The composition of the CoSnC-containing material was analyzed, with the result that the content of tin was 48 mass %, the content of cobalt was 23 mass %, the content of carbon was 20 mass %, and the ratio (Co/(Sn+Co)) of the mass amount of cobalt to the total mass amount of tin and cobalt was 32 mass %.

Next, 80 mass parts of the above-mentioned CoSnC-containing material powder as an anode active material, 12 mass parts of graphite as an electroconductive agent, and 8 mass parts of poly(vinylidene fluoride) as a binding agent were mixed and the resulting mixture was dispersed in N-methyl-2-pyrrolidone. Finally, the resultant material was applied to an anode collector 4A constituted by a copper foil (15 μm thickness), dried and compression molded, to form an anode active material layer 4B. Then, the anode collector 4A having the anode active material layer 4B formed thereon was punched to a circular shape with a diameter of 16 mm. This provided the anode 4.

### Example 3-2

A secondary battery of Example 3-2 was fabricated in the same manner as in Example 3-1 except that Compound 2 was added at 1 wt % instead of addition of Compound 1.

### Example 3-3

A secondary battery of Example 3-3 was fabricated in the same manner as in Example 3-1 except that Compound 3 was added at 1 wt % instead of addition of Compound 1.

### Example 3-4

A secondary battery of Example 3-4 was fabricated in the same manner as in Example 3-1 except that Compound 3 was added at 5 wt % instead of addition of Compound 1.

### Example 3-5

A secondary battery of Example 3-5 was fabricated in the same manner as in Example 3-1 except that Compound 4 was added at 1 wt % instead of addition of Compound 1.

### Example 3-6

A secondary battery of Example 3-6 was fabricated in the same manner as in Example 3-1 except that Compound 5 was added at 1 wt % instead of addition of Compound 1.

### Example 3-7

A secondary battery of Example 3-7 was fabricated in the same manner as in Example 3-1 except that Compound 5 was added at 5 wt % instead of addition of Compound 1.

### Example 3-8

A secondary battery of Example 3-8 was fabricated in the same manner as in Example 3-1 except that Compound 6 was added at 1 wt % instead of addition of Compound 1.

### Example 3-9

A secondary battery of Example 3-9 was fabricated in the same manner as in Example 3-1 except that Compound 6 was added at 5 wt % instead of addition of Compound 1.

### Example 3-10

A secondary battery of Example 3-10 was fabricated in the same manner as in Example 3-1 except that Compound 7 was added at 1 wt % instead of addition of Compound 1.

### Example 3-11

A secondary battery of Example 3-11 was fabricated in the same manner as in Example 3-1 except that Compound 7 was added at 5 wt % instead of addition of Compound 1.

### Example 3-12

A secondary battery of Example 3-12 was fabricated in the same manner as in Example 3-1 except that Compound 8 was added at 1 wt % instead of addition of Compound 1.

### Example 3-13

A secondary battery of Example 3-13 was fabricated in the same manner as in Example 3-1 except that Compound 9 was added at 1 wt % instead of addition of Compound 1.

### Example 3-14

A secondary battery of Example 3-14 was fabricated in the same manner as in Example 3-1 except that Compound 10 was added at 1 wt % instead of addition of Compound 1.

### Example 3-15

A secondary battery of Example 3-15 was fabricated in the same manner as in Example 3-1 except that Compound 12 was added at 1 wt % instead of addition of Compound 1.

### Example 3-16

A secondary battery of Example 3-16 was fabricated in the same manner as in Example 3-1 except that Compound 13 was added at 1 wt % instead of addition of Compound 1.

### Example 3-17

A secondary battery of Example 3-17 was fabricated in the same manner as in Example 3-1 except that Compound 14 was added at 1 wt % instead of addition of Compound 1.

### Example 3-18

A secondary battery of Example 3-18 was fabricated in the same manner as in Example 3-1 except that Compound 15 was added at 1 wt % instead of addition of Compound 1.

### Comparison 3-1

A secondary battery of Comparison 3-1 was fabricated in the same manner as in Example 3-1 except that Compound 1 was not added.

### Comparison 3-2

A secondary battery of Comparison 3-2 was fabricated in the same manner as in Example 3-1 except that Compound 11 was added at 1 wt % instead of addition of Compound 1.

Next, the fabricated secondary batteries of Examples 1-1 to 3-18 and Comparisons 1-1 to 3-2 were, as described below, subjected to the measurement of discharge capacity maintenance rate after 50 cycles and the evaluation of cycle characteristics.

First, the discharge capacity of the second cycle was evaluated by performing two cycles of charge and discharge in an atmosphere of a temperature of 23° C. Subsequently, the discharge capacity of the 50th cycle was evaluated by carrying out 48 cycles of charge and discharge in the same atmosphere. Finally, the discharge capacity maintenance rate was calculated according to the equation: discharge capacity maintenance rate (%)=[(discharge capacity of 50th cycle)/(discharge capacity of second cycle)]×100(%). One cycle of charge and discharge involved charging at a constant current and a constant voltage to a upper limit voltage of 4.2 V at a charging current of 0.2 C and then constant-current-discharging to a final voltage of 2.5 V at a charging current of 0.2 C. In addition, the value 0.2 C refers to a current value of completing discharge (charge) of a theoretical capacity for 5 hours.

Table 1 shows the discharge capacity maintenance rates of Examples 1-1 to 1-14 and Comparisons 1-1 and 1-2. Table 2 shows the discharge capacity maintenance rates of Examples 1-2 and 1-15 to 1-21. Table 3 shows the discharge capacity maintenance rates of Examples 1-2 and 1-22 to 1-25. Table 4 shows the discharge capacity maintenance rates of Examples 2-1 to 2-18 and Comparisons 2-1 and 2-2. Table 5 shows the discharge capacity maintenance rates of Examples 2-19 to 2-30 and Comparisons 2-3 to 2-5. Table 6 shows the discharge capacity maintenance rates of Examples 2-2 and 2-31 to 2-35. Table 7 shows the discharge capacity maintenance rates of Examples 2-2 and 2-36 to 2-39. Table 8 shows the discharge capacity maintenance rates of Examples 3-1 to 3-18 and Comparisons 3-1 and 3-2.

TABLE 1
ANODE: GRAPHITE

| | | | DISCHARGE |
|---|---|---|---|
| | | ADDITION | CAPACITY |
| | | AMOUNT | MAINTENANCE |
| | ADDITIVE | [wt %] | RATE [%] |
| EXAMPLE 1-1 | COMPOUND 1 | 1 | 83 |
| EXAMPLE 1-2 | COMPOUND 2 | 1 | 88 |
| EXAMPLE 1-3 | COMPOUND 3 | 1 | 87 |
| EXAMPLE 1-4 | COMPOUND 4 | 1 | 86 |
| EXAMPLE 1-5 | COMPOUND 5 | 1 | 85 |
| EXAMPLE 1-6 | COMPOUND 6 | 1 | 89 |
| EXAMPLE 1-7 | COMPOUND 7 | 1 | 92 |
| EXAMPLE 1-8 | COMPOUND 8 | 1 | 91 |
| EXAMPLE 1-9 | COMPOUND 9 | 1 | 89 |
| EXAMPLE 1-10 | COMPOUND 10 | 1 | 84 |
| EXAMPLE 1-11 | COMPOUND 12 | 1 | 87 |
| EXAMPLE 1-12 | COMPOUND 13 | 1 | 88 |
| EXAMPLE 1-13 | COMPOUND 14 | 1 | 90 |
| EXAMPLE 1-14 | COMPOUND 15 | 1 | 91 |
| COMPARISON 1-1 | — | — | 76 |
| COMPARISON 1-2 | COMPOUND 11 | 1 | 80 |

TABLE 2
ANODE: GRAPHITE

| | | | | | DISCHARGE |
|---|---|---|---|---|---|
| | | ADDITION | | ADDITION | CAPACITY |
| | | AMOUNT | | AMOUNT | MAINTENANCE |
| | ADDITIVE | [wt %] | ADDITIVE | [wt %] | RATE [%] |
| EXAMPLE 1-15 | COMPOUND 2 | 1 | VC | 1 | 90 |
| EXAMPLE 1-16 | COMPOUND 2 | 1 | FEC | 1 | 91 |
| EXAMPLE 1-17 | COMPOUND 2 | 1 | DFEC | 1 | 93 |
| EXAMPLE 1-18 | COMPOUND 2 | 1 | PRS | 1 | 89 |
| EXAMPLE 1-19 | COMPOUND 2 | 1 | SUCCINIC | 1 | 91 |
| | | | ANHYDRIDE | | |
| EXAMPLE 1-20 | COMPOUND 2 | 1 | 2-SULFOBENZOIC | 1 | 91 |
| | | | ANHYDRIDE | | |
| EXAMPLE 1-21 | COMPOUND 2 | 1 | ETHYLENE SULFITE | 1 | 86 |
| EXAMPLE 1-2 | COMPOUND 2 | 1 | — | — | 88 |

VC: VINYLENE CARBONATE
FEC: 4-FLUORO-1,3-DIOXOLANE-2-ONE
DFEC: 4,5-DIFLUORO-1,3-DIOXOLANE-2-ONE
PRS: PROPENE SULTONE

TABLE 3
ANODE: GRAPHITE

| | | | | | | DISCHARGE |
|---|---|---|---|---|---|---|
| | | ADDITION | | | ADDITION | CAPACITY |
| | | AMOUNT | ELECTROLYTE | ELECTROLYTE | AMOUNT | MAINTENANCE |
| | ADDITIVE | [wt %] | SALT | SALT | [mol/kg] | RATE [%] |
| EXAMPLE 1-22 | COMPOUND 2 | 1 | LiPF₆ | LiBF₄ | 0.1 | 89 |
| EXAMPLE 1-23 | COMPOUND 2 | 1 | (1 mol/kg) | LiBOB | 0.1 | 90 |
| EXAMPLE 1-24 | COMPOUND 2 | 1 | | LiTFSI | 0.1 | 90 |
| EXAMPLE 1-25 | COMPOUND 2 | 1 | | CYCLIC IMIDE | 0.1 | 89 |
| EXAMPLE 1-2 | COMPOUND 2 | 1 | | — | — | 88 |

LiBOB: LITHIUM BISOXALATE BORATE
LiTFSI: LITHIUM BIS (TRIFLUOROMETHANESULFONYL) IMIDE
CYCLIC IMIDE: LITHIUM 1,3-PERFLUOROPROPANEDISULFONYLIMIDE

TABLE 4
ANODE: SILICON (Si)

| | | | DISCHARGE |
|---|---|---|---|
| | | ADDITION | CAPACITY |
| | | AMOUNT | MAINTENANCE |
| | ADDITIVE | [wt %] | RATE [%] |
| EXAMPLE 2-1 | COMPOUND 1 | 1 | 68 |
| EXAMPLE 2-2 | COMPOUND 2 | 1 | 70 |
| EXAMPLE 2-3 | COMPOUND 3 | 1 | 68 |
| EXAMPLE 2-4 | COMPOUND 3 | 5 | 68 |
| EXAMPLE 2-5 | COMPOUND 4 | 1 | 65 |
| EXAMPLE 2-6 | COMPOUND 5 | 1 | 63 |
| EXAMPLE 2-7 | COMPOUND 5 | 5 | 64 |
| EXAMPLE 2-8 | COMPOUND 6 | 1 | 75 |
| EXAMPLE 2-9 | COMPOUND 6 | 5 | 78 |
| EXAMPLE 2-10 | COMPOUND 7 | 1 | 85 |
| EXAMPLE 2-11 | COMPOUND 7 | 5 | 87 |
| EXAMPLE 2-12 | COMPOUND 8 | 1 | 88 |
| EXAMPLE 2-13 | COMPOUND 9 | 1 | 71 |
| EXAMPLE 2-14 | COMPOUND 10 | 1 | 63 |
| EXAMPLE 2-15 | COMPOUND 12 | 1 | 66 |
| EXAMPLE 2-16 | COMPOUND 13 | 1 | 69 |
| EXAMPLE 2-17 | COMPOUND 14 | 1 | 80 |
| EXAMPLE 2-18 | COMPOUND 15 | 1 | 85 |
| COMPARISON 2-1 | — | — | 45 |
| COMPARISON 2-2 | COMPOUND 11 | 1 | 50 |

TABLE 5
ANODE: SILICON (Si)

| | | | | | | | DISCHARGE |
|---|---|---|---|---|---|---|---|
| | SOLVENT | | | | | ADDITION | CAPACITY |
| | (WEIGHT RATIO) | | | | | AMOUNT | MAINTENANCE |
| | PC | FEC | DFEC | DEC | ADDITIVE | [wt %] | RATE [%] |
| EXAMPLE 2-19 | — | 50 | — | 50 | COMPOUND 2 | 1 | 85 |
| EXAMPLE 2-20 | — | 50 | — | 50 | COMPOUND 6 | 1 | 88 |
| EXAMPLE 2-21 | — | 50 | — | 50 | COMPOUND 7 | 1 | 89 |
| EXAMPLE 2-22 | — | 50 | — | 50 | COMPOUND 8 | 1 | 89 |
| EXAMPLE 2-23 | 40 | — | 10 | 50 | COMPOUND 2 | 1 | 87 |
| EXAMPLE 2-24 | 40 | — | 10 | 50 | COMPOUND 6 | 1 | 89 |
| EXAMPLE 2-25 | 40 | — | 10 | 50 | COMPOUND 7 | 1 | 90 |
| EXAMPLE 2-26 | 40 | — | 10 | 50 | COMPOUND 8 | 1 | 89 |
| EXAMPLE 2-27 | 30 | 10 | 10 | 50 | COMPOUND 2 | 1 | 89 |
| EXAMPLE 2-28 | 30 | 10 | 10 | 50 | COMPOUND 6 | 1 | 90 |
| EXAMPLE 2-29 | 30 | 10 | 10 | 50 | COMPOUND 7 | 1 | 92 |
| EXAMPLE 2-30 | 30 | 10 | 10 | 50 | COMPOUND 8 | 1 | 91 |
| COMPARISON 2-3 | — | 50 | — | 50 | — | — | 80 |
| COMPARISON 2-4 | 40 | — | 10 | 50 | — | — | 81 |
| COMPARISON 2-5 | 30 | 10 | 10 | 50 | — | — | 83 |

PC: PROPYLENE CARBONATE
FEC: 4-FLUORO-1,3-DIOXOLANE-2-ONE
DFEC: 4,5-DIFLUORO-1,3-DIOXOLANE-2-ONE
DEC: DIETHYLENE CARBONATE

TABLE 6
ANODE: SILICON (Si)

| | | | | | DISCHARGE |
|---|---|---|---|---|---|
| | | ADDITION | | ADDITION | CAPACITY |
| | | AMOUNT | | AMOUNT | MAINTENANCE |
| | ADDITIVE | [wt %] | ADDITIVE | [wt %] | RATE [%] |
| EXAMPLE 2-31 | COMPOUND 2 | 1 | VC | 1 | 73 |
| EXAMPLE 2-32 | COMPOUND 2 | 1 | PRS | 1 | 71 |
| EXAMPLE 2-33 | COMPOUND 2 | 1 | SUCCINIC | 1 | 72 |
| | | | ANHYDRIDE | | |
| EXAMPLE 2-34 | COMPOUND 2 | 1 | 2-SULFOBENZOIC | 1 | 73 |
| | | | ANHYDRIDE | | |
| EXAMPLE 2-35 | COMPOUND 2 | 1 | ETHYLENE | 1 | 65 |
| | | | SULFITE | | |
| EXAMPLE 2-2 | COMPOUND 2 | 1 | — | 1 | 70 |

VC: VINYLENE CARBONATE
PRS: PROPENE SULTONE

TABLE 7
ANODE: SILICON (Si)

| | | | | | | DISCHARGE |
|---|---|---|---|---|---|---|
| | | ADDITION | | | ADDITION | CAPACITY |
| | | AMOUNT | ELECTROLYTE | ELECTROLYTE | AMOUNT | MAINTENANCE |
| | ADDITIVE | [wt %] | SALT | SALT | [mol/kg] | RATE [%] |
| EXAMPLE 2-36 | COMPOUND 2 | 1 | LiPF₆ | LiBF₄ | 0.1 | 70 |
| EXAMPLE 2-37 | COMPOUND 2 | 1 | (1 mol/kg) | LiBOB | 0.1 | 70 |
| EXAMPLE 2-38 | COMPOUND 2 | 1 | | LiTFSI | 0.1 | 72 |
| EXAMPLE 2-39 | COMPOUND 2 | 1 | | CYCLIC IMIDE | 0.1 | 71 |
| EXAMPLE 2-2 | COMPOUND 2 | 1 | | — | 0.1 | 70 |

LiBOB: LITHIUM BISOXALATE BORATE
LiTFSI: LITHIUM BIS (TRIFLUOROMETHANESULFONYL) IMIDE
CYCLIC IMIDE: LITHIUM 1,3-PERFLUOROPROPANEDISULFONYLIMIDE

TABLE 8
ANODE: CoSnC-CONTAINING MATERIAL

| | | | DISCHARGE |
|---|---|---|---|
| | | ADDITION | CAPACITY |
| | | AMOUNT | MAINTENANCE |
| | ADDITIVE | [wt %] | RATE [%] |
| EXAMPLE 3-1 | COMPOUND 1 | 1 | 63 |
| EXAMPLE 3-2 | COMPOUND 2 | 1 | 65 |
| EXAMPLE 3-3 | COMPOUND 3 | 1 | 66 |
| EXAMPLE 3-4 | COMPOUND 3 | 5 | 64 |
| EXAMPLE 3-5 | COMPOUND 4 | 1 | 63 |
| EXAMPLE 3-6 | COMPOUND 5 | 1 | 63 |
| EXAMPLE 3-7 | COMPOUND 5 | 5 | 64 |
| EXAMPLE 3-8 | COMPOUND 6 | 1 | 70 |
| EXAMPLE 3-9 | COMPOUND 6 | 5 | 71 |
| EXAMPLE 3-10 | COMPOUND 7 | 1 | 83 |
| EXAMPLE 3-11 | COMPOUND 7 | 5 | 84 |
| EXAMPLE 3-12 | COMPOUND 8 | 1 | 83 |
| EXAMPLE 3-13 | COMPOUND 9 | 1 | 70 |
| EXAMPLE 3-14 | COMPOUND 10 | 1 | 61 |
| EXAMPLE 3-15 | COMPOUND 12 | 1 | 65 |
| EXAMPLE 3-16 | COMPOUND 13 | 1 | 67 |
| EXAMPLE 3-17 | COMPOUND 14 | 1 | 76 |
| EXAMPLE 3-18 | COMPOUND 15 | 1 | 83 |
| COMPARISON 3-1 | — | — | 50 |
| COMPARISON 3-2 | COMPOUND 11 | 1 | 53 |

As shown in Table 1, Examples 1-1 to 1-14 exhibited high discharge capacity maintenance rates as compared with Comparison 1-1 containing no additive and Comparison 1-2 containing Compound 11. In other words, it has been found that in the case of using a carbon material for an anode, inclusion of the compound represented by Chemical formula 3 in an electrolyte solution enables the improvement of cycle characteristics.

In addition, among Examples 1-1 to 1-8, Examples 1-6 to 1-8 containing as electrolyte solutions compounds in which R1 and R2 each represent an allyl group, a trimethylsilyl group or a fluorine-substituted hydrocarbon group exhibited high discharge capacity maintenance rates. That is to say, it has been found that in the case where a carbon material is used for an anode, inclusion of, in an electrolyte solution, the compound represented by Chemical formula 3 in which R1 and R2 each represent an allyl group, a trialkylsilyl group or a fluorine-substituted hydrocarbon group enables the improvement of cycle characteristics.

As shown in Table 2, Examples 1-15 to 1-20 showed high discharge capacity maintenance rates as compared with Example 1-2. Additionally, Example 1-21 exhibited a low discharge capacity maintenance rate compared with Example 1-2. In other words, it has been found that in the case of using a carbon material for an anode, the compound represented by Chemical formula 3 can be used without deterioration of cycle characteristics in combination with vinylene carbonate (VC), 4-fluoro-1,3-dioxolane-2-one (FEC), 4,5-difluoro-1,3-dioxolane-2-one (DFEC), propene sultone (PRS), succinic anhydride or 2-sulfobenzoic anhydride.

As indicated in Table 3, Examples 1-22 to 1-25 showed high discharge capacity maintenance rates compared with Example 1-2. In other words, it has been found that in the case where a carbon material is used for an anode, use of the compound represented by Chemical formula 3 in combination with LiBF4, lithium bisoxalate borate (LiBOB), lithium bis(trifluoromethanesulfonyl)imide (LiTFSI) or lithium 1,3-perfluoropropanedisulfonylimide, as an electrolyte salt, further enables the improvement of cycle characteristics.

As shown in Table 4, Examples 2-1 to 2-18 exhibited high discharge capacity maintenance rates as compared with Comparison 2-1 containing no additive and Comparison 1-2 containing Compound 11. In other words, it has been found that in the case where a material containing silicon Si as a constituent element is used for an anode, inclusion of the compound represented by Chemical formula 3 in an electrolyte solution enables the improvement of cycle characteristics.

Additionally, among Examples 2-1 to 2-12, Examples 2-8 to 2-12 containing as electrolyte solutions compounds represented by Chemical formula 3 in which R1 and R2 each represent an allyl group, a trimethylsilyl group or a fluorine-substituted hydrocarbon group particularly exhibited high discharge capacity maintenance rates. That is to say, it has been found that in the case where a material containing silicon Si as a constituent element is used for an anode, inclusion of, in an electrolyte solution, the compound represented by Chemical formula 3 in which R1 and R2 each represent an allyl group, a trialkylsilyl group or a fluorine-substituted hydrocarbon group enables the improvement of cycle characteristics.

As shown in Table 5, Examples 2-19 to 2-22 showed high discharge capacity maintenance rates as compared with Comparison 2-3. Examples 2-23 to 2-26 showed high discharge capacity maintenance rates as compared with Comparison 2-4. Examples 2-27 to 2-30 showed high discharge capacity maintenance rates as compared with Comparison 2-5.

As shown in Table 6, Examples 2-31 to 2-34 showed high discharge capacity maintenance rates as compared with Example 2-2. Additionally, Example 2-35 exhibited a low discharge capacity maintenance rate compared with Example 2-2. In other words, it has been found that in the case where a material containing silicon Si as a constituent element is used for an anode, use of the compound represented by Chemical formula 3 in combination with vinylene carbonate (VC), 4-fluoro-1,3-dioxolane-2-one (FEC), 4,5-difluoro-1,3-dioxolane-2-one (DFEC), propene sultone (PRS), succinic anhydride or 2-sulfobenzoic anhydride enables the improvement of cycle characteristics.

As shown in Table 7, Examples 2-36 to 2-39 exhibited discharge capacity maintenance rates equivalent to or higher than that of Example 2-2. In other words, it has been found that in the case where a material containing silicon Si as a constituent element is used for an anode, the compound represented by Chemical formula 3 can be used without deterioration of cycle characteristics in combination with LiBF4, lithium bisoxalate borate (LiBOB), lithium bis(trifluoromethanesulfonyl)imide (LiTFSI) or lithium 1,3-perfluoropropanedisulfonylimide, as an electrolyte salt.

As shown in Table 8, Examples 3-1 to 3-18 exhibited high discharge capacity maintenance rates as compared with Comparison 3-1 containing no additive and Comparison 3-2 containing Compound 11. In other words, it has been found that in the case of using a material containing tin Sn as a constituent element for an anode, inclusion of the compound represented by Chemical formula 3 enables the improvement of cycle characteristics.

Among Examples 3-1 to 3-12, Examples 3-8 to 3-12 containing as electrolyte solutions compounds represented by Chemical formula 3 in which R1 and R2 each represent an allyl group, a trimethylsilyl group or a fluorine-substituted hydrocarbon group particularly exhibited high discharge capacity maintenance rates. That is to say, it has been found that in the case where a material containing tin Sn as a constituent element is used for an anode, inclusion of, in an electrolyte solution, the compound represented by Chemical formula 3 in which R1 and R2 each represent an allyl group, a trialkylsilyl group or a fluorine-substituted hydrocarbon group enables the improvement of cycle characteristics.

In addition, as indicated in Tables 1 to 8, the case where a material containing silicon Si as a constituent element was used for an anode or where a material containing tin Sn as a constituent element was used for an anode showed to obtain a higher effect of cycle characteristics than the case where a carbon material was used for an anode.

Additionally, in the examples described above, the case where a non-aqueous electrolyte solution is used as a non-aqueous electrolyte has been described. However, a similar result tends to be obtained even in the case of using a gel-like non-aqueous electrolyte.

It should be understood by those skilled in the art that various modifications, combinations, sub-combinations and alternations may occur depending on design requirements and other factors insofar as they are within the scope of the appended claims or the equivalents thereof. For example, in the embodiments described above, examples including a coin-type battery, a cylindrical battery, a flat-type battery using a laminate or the like for an outer covering material have been described, but the invention is by no means limited to these. The embodiments of the invention can also include, for example, various kinds, shapes or sizes of batteries such as an angular battery.

Moreover, in the embodiments described above, a lithium ion secondary battery has been described by way of example, and the embodiments of this invention can be applied to other secondary batteries. Examples of the other secondary batteries include a so-called metal lithium secondary battery using metal lithium as an anode active material, and a magnesium secondary battery or aluminum secondary battery, which has been studied for commercialization. Furthermore, the embodiments can be applied not only to a battery involving chemical reaction, but to other electrochemical devices such as an electric double-layer capacitor using an electrolyte solution.

Still furthermore, as the non-aqueous electrolyte, for example, a polymer solid electrolyte making use of an ion conducting polymer or an inorganic solid electrolyte utilizing an ion conducting inorganic material may be used. These may be used alone or in combination with another electrolyte. Examples of the polymer compounds that can be used for a polymer solid electrolyte include polyether, polyester, polyphosphazene, and polysiloxane. Examples of the inorganic solid electrolytes include ion conducting ceramics, ion conducting crystals, and ion conducting glass.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A non-aqueous electrolyte comprising:
at least one kind selected from the compounds represented by Chemical formula 1: 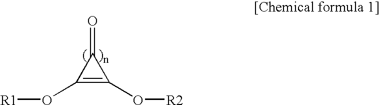
wherein R1 and R2 each represent a hydrogen group, a hydrocarbon group or an alkylsilyl group, or any one group thereof where a portion, part or all hydrogen atoms of which is substituted with a halogen, R1 and R2 may be bonded to each other, and n is an integer of 1 or larger.

2. The non-aqueous electrolyte according to claim 1, wherein the compound represented by Chemical formula 1 is a compound wherein n is from 1 to 6 in Chemical formula 1.

3. The non-aqueous electrolyte according to claim 1, wherein the compound represented by Chemical formula 1 is a compound of Chemical formula 1 in which R1 and R2 each represent an allyl group or a trialkylsilyl group, or any group thereof where a portion or all of the hydrogen atoms of which is/are substituted with fluorine.

4. The non-aqueous electrolyte according to claim 1, wherein the compound represented by Chemical formula 1 is squaric acid or a squaric acid derivative represented by Chemical formula 2: 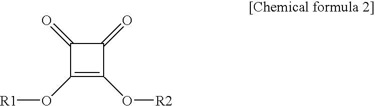
wherein R1 and R2 each represent a hydrogen group, a hydrocarbon group or an alkylsilyl group, or any group thereof where a portion or all hydrogen atoms of which is substituted with a halogen, and R1 and R2 may be bonded to each other.

5. The non-aqueous electrolyte according to claim 1, further comprising at least one compound selected from 4-fluoro-1,3-dioxolane-2-one and 4,5-difluoro-1,3-dioxolane-2-one.

6. The non-aqueous electrolyte according to claim 1, further comprising a cyclic sultone derivative.

7. The non-aqueous electrolyte according to claim 1, further comprising an acid anhydride.

8. The non-aqueous electrolyte according to claim 1, further comprising a cyclic carbonate ester compound having an unsaturated bond.

9. A non-aqueous electrolyte battery comprising:
a cathode;
an anode; and
a non-aqueous electrolyte,
wherein the non-aqueous electrolyte includes at least one compound represented by Chemical formula 3: 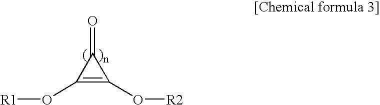
wherein R1 and R2 each represent a hydrogen group, a hydrocarbon group or an alkylsilyl groups, or any group thereof where a portion or all hydrogen atoms of which is substituted with a halogen, R1 and R2 may be bonded to each other, and n is an integer of 1 or larger.

10. The non-aqueous electrolyte battery according to claim 9, wherein
the compound represented by Chemical formula 3 is a compound wherein n is from 1 to 6 in Chemical formula 3.

11. The non-aqueous electrolyte battery according to claim 9, wherein
the compound represented by Chemical formula 3 is a compound of Chemical formula 3 in which R1 and R2 each represent an allyl group or a trialkylsilyl group, or any of these groups part or all of the hydrogen atoms of which is/are substituted with fluorine.

12. The non-aqueous electrolyte battery according to claim 9, wherein
the compound represented by Chemical formula 1 is squaric acid or a squaric acid derivative represented by Chemical formula 4: 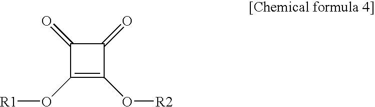
wherein R1 and R2 each represent a hydrogen group, a hydrocarbon group or an alkylsilyl group, or any group thereof where a portion or all hydrogen atoms of which is substituted with a halogen, and R1 and R2 may be bonded to each other.

13. The non-aqueous electrolyte battery according to claim 9, wherein
the anode contains a material including as a constituent element at least one of silicon and tin.
